(19) United States
(12) Patent Application Publication
(10) Pub. No.: US 2021/0145763 A1
(43) Pub. Date: May 20, 2021

(54) LOW THC HEMP EXTRACT AND METHOD OF TREATMENT OR PREVENTION OF AN EYE DISEASE

(71) Applicant: EYE CO PTY LTD, Balwyn North, Victoria (AU)

(72) Inventor: Philip Leslie Penfold, Balwyn North, Victoria (AU)

(21) Appl. No.: 17/044,885

(22) PCT Filed: Apr. 4, 2019
(86) PCT No.: PCT/AU2019/000041

§ 371 Date: Oct. 2, 2020

(30) Foreign Application Priority Data
Apr. 4, 2018 (AU) 2018901116

(51) Int. Cl. A61K 31/05; A61K 36/185; A61K 31/20; A61K 9/00; A61P 27/02; A61K 45/06

(52) U.S. Cl. CPC A61K 31/05; A61K 36/185; A61K 31/20; A61K 2236/31; A61K 9/0048; A61P 27/02; A61K 45/06; A61K 9/0019

(57) ABSTRACT

The present invention relates to methods of treating or preventing AMD in a subject, the method comprising administering to the subject a therapeutically effective amount of hemp, hemp oil or pharmaceutically effective extract thereof to thereby treat or prevent the AMD. The invention also relates to pharmaceutical composition comprising a therapeutically effective amount of hemp, hemp oil; or a pharmaceutically effective extract thereof and a pharmaceutically acceptable carrier, diluent or excipient when used to treat AMD, and to uses of a pharmaceutical composition comprising hemp, hemp oil, or a pharmaceutically effective extract thereof for the manufacture of a medicament for the treatment of AMD.

### FIELD OF THE INVENTION

The present invention relates to a composition and method of treatment for age related macular degeneration (A.M.D. or AMD). More particularly, this invention relates to a composition and method of treatment or prevention of AMD comprising hemp, hemp oil or a pharmaceutically active extract thereof.

### BACKGROUND TO THE INVENTION

In Australia, one in seven individuals over the age of 50 present with symptoms of Age-Related Macular Degeneration (AMD). Definitive causes heralding the onset of AMD remains elusive, as does a lack of treatment options for patients with non-neovascular manifestations of the disease. It is widely accepted that there is no medical or surgical treatment for AMD and there is currently no proven treatment for Dry Macular Degeneration (d AMD) or the end stage Geographic Atrophy (GA).

Macular Degeneration is the world's leading cause of blindness costing the Australian economy alone $5 Billion per annum. Current projections indicate that by 2030, ˜1.7 million Australians will suffer vision loss due to AMD. A major contributing factor to this dire outlook is the lack of early diagnostics for onset of disease, prior to overt pathological changes in the retina. Currently, early AMD is characterised clinically by the appearance of pigmentary changes in the retinal pigment epithelium (RPE) and the presence of drusen, non-degrading sub-retinal deposits comprising a complex of proteins and oxidised lipids. Early AMD can progress to two advanced forms; geographic atrophy (GA/‘dry’ AMD), or choroidal neovascularisation (CNV/‘wet’ AMD). GA is an advanced form of AMD that can result in the progressive and irreversible atrophy of retina (photoreceptors, retinal pigment epithelium (RPE) and choriocappillaris). The pathogenesis of GA is multifactorial and is thought to be triggered by intrinsic and extrinsic stressors of the poorly regenerative RPE. The regions of atrophy can look like a map, this explains the term “geographic”. The term GA is used interchangeably with the term “AMD”.

Regardless of the pathology, in late stage AMD, patients are seriously impacted by central vision loss, resulting from photoreceptor and RPE cell death. Oxidative stress and inflammation are heavily implicated in both early and late stages of AMD pathogenesis.

Aruna Gorusupudi, Kelly Nelson, and Paul S Bernstein noted that a wide variety of nutrients, such as minerals, vitamins, v-3 (n-3) fatty acids, and various carotenoids, have been associated with reducing the risk of AMD. These authors noted that results from the Age-Related Eye Disease Study (AREDS) indicated that supplementation with antioxidants (b-carotene and vitamins C and E) and zinc was associated with a reduced risk of AMD progression and that the AREDS2 follow-up study, was designed to improve upon the earlier formulation, tested the addition of lutein, zeaxanthin, and v-3 fatty acids.

However, Singh states that AREDS failed to show that vitamin supplementation decreased progression to GA. Even in AREDS2, when beta-carotene was replaced with lutein/zeaxanthin to decrease the risk of lung cancer, the new formulation also failed to show decreased progression to GA. Singh refers to clinical studies underway at Cole Eye Institute, Cleveland Clinic, to further elucidate and understand the mechanisms of dry AMD and to evaluate new therapeutics directed at slowing the progression. There are two large phase 3 trials underway for the treatment of GA. The FILLY study assesses the safety, tolerability and evidence of activity of multiple intravitreal (IVT) injections of APL-2 (Apellis Pharmaceuticals) for patients with GA. The second is a multicenter, randomized, double-masked, sham-controlled study to investigate IVT injections of lampalizumab in patients with GA.

Singh also refers to another area of research that has sprung from the discovery of complement by-products in drusen which led to associations between complement dysregulation and AMD. Several researchers are now evaluating the complement cascade as a clinical therapeutic target for non-neovascular AMD. Factor D is considered an early component of the alternative pathway that involves complement factor H. Anti-inflammatory agents under development or previously under development include lampalizumab, fluocinolone, glatiramer acetate, sirolimus, eculizumab and ARC-1905.

Hemp, including low Δ⁶ tetrahydrocannabinol hemp, has been investigated for therapeutic activity. The University of Wollongong School and Creso Pharma are conducting a study with an emphasis on investigating how cannabidiol, a non-intoxicating component of cannabis, influences learning, memory and attention, and has potential for a wide variety of conditions including schizophrenia.

U.S. Pat. No. 5,521,215 describes pharmaceutical compositions for preventing neurotoxicity, comprising as the active ingredient the stereospecific (+) enantiomer, having (3S,4S) configuration of Δ⁶ tetrahydrocannabinol type compounds. The compositions were described as being particularly effective in alleviating and even preventing neurotoxicity due to acute injuries to the central nervous system, including mechanical trauma, compromised or reduced blood supply as may occur in cardiac arrest or stroke, or poisonings. They were also described as effective in the treatment of certain chronic degenerative diseases characterized by gradual neuronal loss.

There remains a need for treatments for AMD and the progression thereof.

The reference to any prior art in this specification is not, and should not be taken as, an acknowledgement or any form of suggestion that the prior art forms part of the common general knowledge.

### SUMMARY OF THE INVENTION

Generally, embodiments of the present invention relate to a composition and method for treatment or prevention of age related macular degeneration (AMD).

In a broad form, the invention relates to use of a hemp, hemp oil or a pharmaceutically effective extract thereof in the treatment of AMD.

In one aspect, although it need not be the only or indeed the broadest form, the invention provides a method of treating or preventing AMD in a subject, the method comprising administering to the subject a therapeutically effective amount of hemp, hemp oil or pharmaceutically effective extract thereof to thereby treat or prevent the AMD.

In a second aspect, the invention provides a pharmaceutical composition comprising a therapeutically effective amount of hemp, hemp oil; or a pharmaceutically effective extract thereof and a pharmaceutically acceptable carrier, diluent or excipient when used to treat AMD.

In a third aspect, the invention provides use of a pharmaceutical composition comprising hemp, hemp oil; or a pharmaceutically effective extract thereof for the manufacture of a medicament for the treatment of AMD.

In one embodiment of any one of the above aspects, the hemp, hemp oil or pharmaceutically effective extract thereof comprises a cannabinoid. The cannabinoid may comprise cannabidiol.

In another embodiment of any one of the above aspects, the hemp, hemp oil or pharmaceutically active extract thereof may comprise a low Tetrahydrocannabinol (THC) hemp, hemp oil or pharmaceutically effective extract thereof.

In another embodiment of any one of the above aspect, the hemp, hemp oil or pharmaceutically effective extract may comprise a Cannabis Ruderalis.

In yet another embodiment of any one of the above aspects, the composition comprises a water-soluble dosage form.

In another embodiment of any one of the above aspects, the hemp oil may be obtained from hemp seeds.

In another embodiment of any one of the above aspects, the hemp oil may be cold-pressed.

In another embodiment of any one of the above aspects, the hemp oil may comprise about 80% to 90% balanced Omega fatty acids. That is, hemp oil comprises Omega 3, (ALA), Omega 6 (LA), Omega 6 (GLA), and Omega 9 (oleic acid), which in combination may amount to 80% to 90% of the composition of the hemp oil.

The hemp oil may comprise about 88% balanced Omega fatty acids. That is, the hemp oil may comprise about 88 g Omega fatty acids per 100 g of hemp oil.

The hemp oil may comprise about 15% to 25% Omega 3, (ALA), about 50% to 60% Omega 6 (LA), about 1% to 5% Omega 6 (GLA), and about 10% to 15% Omega 9 (oleic acid), per 100 g of hemp oil.

The hemp oil may comprise about 1 g to 5 g Omega 3, (ALA), about 5 g to 15 g Omega 6 (LA), about 0.2 g to 1 g Omega 6 (GLA), and about 1 g to 5 g Omega 9 (oleic acid), per 20 g of hemp oil.

In some embodiments of any one of the above aspects, the hemp oil may comprise about 3.5 g Omega 3, (ALA), about 11.2 g Omega 6 (LA), about 0.4 g Omega 6 (GLA), and about 2.5 g Omega 9 (oleic acid).

In some embodiments of any one of the above aspects, the hemp oil may comprise about 3.3 g Omega 3, (ALA), about 10.7 g Omega 6 (LA), about 0.7 g Omega 6 (GLA), and about 2.7 g Omega 9 (oleic acid).

In another embodiment of any one of the above aspects, the hemp oil may have a ratio of Omega 3 to Omega 6 of between about 1:5.2 and 5:16. The hemp oil may have a ratio of Omega 3 to Omega 6 of about 3.5:11.6.

In another embodiment of any one of the above aspects, the pharmaceutical composition may be for use or when used as a carrier or delivery vehicle for one or more compounds. The one or more compounds may be pharmaceutically active. The one or more compounds may comprise an hydrophobic compound.

In still another embodiment, the hemp, hemp oil; therapeutically effective amount or pharmaceutical composition comprises a form suitable for administration by one or more of intradermal, intramuscular, intraperitoneal, parenteral, intravenous, subcutaneous, intranasal, epidural, sublingual, intracerebral, intravaginal, transdermal (e.g., via a patch), rectal, by inhalation, transmucosal, or topical, particularly to the ears, nose, eyes, or skin. The pharmaceutical composition may be injectable. The parenteral or injectable form may comprise any suitable form for parenteral or injectable administration such as an injectable solution, an injectable suspension, an injectable emulsion, and an injection in a form that is prepared at the time of use. Formulations for parenteral administration may be in a configuration such as an aqueous or nonaqueous isotonic aseptic solution or suspension. The injectable form may be for intravitreal injection.

In another particular embodiment of any above aspect, the pharmaceutical composition is preservative free.

In another particular embodiment of any above aspect, the pharmaceutical composition may be prophylactic.

According to any one of the above aspects, the pharmaceutical composition of the invention may comprise a sustained release composition.

In a particular embodiment of any one of the above aspects, the pharmaceutical composition or one or more component thereof may be sterilized.

In another embodiment of any above aspect, the hemp seed oil may function as a carrier for one or more compounds.

The one or more compounds may comprise an anti-inflammatory compound. The anti-inflammatory compound may comprise one or more of a COX inhibitor, one or more mineralocorticoid or a therapeutically active analogue, derivative, homolog, pharmaceutically acceptable salt or conjugate thereof, one or more glucocorticoid or a therapeutically active analogue, derivative, homolog, pharmaceutically acceptable salt or conjugate thereof, an antileukotrine and/or a leukotriene receptor antagonist. The COX inhibitor may inhibit one or both of COX-1 and COX-2. The COX inhibitor may comprise a Non-Steroidal Anti-Inflammatory Drug (NSAID). The NSAID may comprise ibuprofen, copper ibuprofenate, indomethacin, copper indomethacin, naproxen, flurbiprofen and/or celecoxib.

According to any above aspect, the one or more anti-inflammatory may for example comprise one or more of: aceclofenac, acemetacin, acetylsalicylic acid, 5-amino-acetylsalicylic acid, alclofenac, alminoprofen, amfenac, bendazac, bermoprofen, alpha-bisabolol, bromfenac, bromosaligenin, bucloxic acid, butibufen, carprofen, cinmetacin, clidanac, clopirac, diclofenac sodium, diflunisal, ditazol, enfenamic acid, etodolac, etofenamate, felbinac, fenbufen, fenclozic acid, fendosal, fenoprofen, fentiazac, fepradinol, flufenamic acid, flunixin, flunoxaprofen, flurbiprofen, glucametacin, glycol salicylate, ibuprofen, ibuproxam, indomethacin, indoprofen, isofezolac, isoxepac, isoxicam, ketoprofen, ketorolac, lornoxicam, loxoprofen, meclofenamic acid, mefenamic acid, meloxicam, mesalamine, metiazinic acid, mofezolac, naproxen, niflumic acid, oxaceprol, oxaprozin, oxyphenbutazone, parsalmide, perisoxal, phenyl acetylsalicylate, olsalazine, pyrazolac, piroxicam, pirprofen, pranoprofen, protizinic acid, salacetamide, salicilamide O-acetic acid, salicylsulphuric acid, salsalate, sulindac, suprofen, suxibuzone, tenoxicam, tiaprofenic acid, tiaramide, tinoridine, tolfenamic acid, tolmetin, tropesin, xenbucin, ximoprofen, zaltoprofen, zomepirac, tomoxiprol; and sulindac.

The one or more compounds may comprise one or more of: 11-desoxycortisone (11-DC); fludrocortisone; fludrocortisone acetate (FA); fludrocortisone acetonide; Deoxycorticosterone acetate (DA); Deoxycorticosterone (DS); Aldosterone; cortisol, cortisone, prednisone, prednisolone, methylprednisolone, dexamethasone, betamethasone, triamcinolone, triamcinolone acetonide, or beclometasone or a therapeutically active analogue, derivative, homolog, pharmaceutically acceptable salt or conjugate thereof.

The one or more mineralocorticoid and/or more glucocorticoid or a therapeutically active analogue, derivative, homolog, pharmaceutically acceptable salt or conjugate thereof may comprise one or more dual action compounds, wherein each dual action compound is capable of modulating the activity of both a mineralocorticoid receptor and a glucocorticoid receptor.

The dual action compound may comprise one or more of triamcinolone; triamcinolone acetonide; cortisol; cortisone; prednisone; prednisolone; methylprednisolone; fludrocortisone; fludrocortisone acetate; fludrocortisone acetonide; or a therapeutically active analogue, derivative, homolog, pharmaceutically acceptable salt or conjugate thereof.

In one embodiment of any one of the above aspects, the method further comprises administering to the subject at least one additional agent. The additional agent may comprise one or more Omega-3 Fatty Acids. The additional agent may comprise an anti-VEGF (anti-Vascular Endothelial Growth Factor). The anti-VEGF may comprise one or more of ranibizumab (brand name Lucentis®); aflibercept (brand name Eylea®); bevacizumab (brand name Avastin®) and OPT-302.

The Applicant is also the owner of PCT/AU2019/000023, the disclosure of which is incorporated herein by reference.

Further aspects and/or features of the present invention will become apparent from the following detailed description.

### BRIEF DESCRIPTION OF THE DRAWINGS

In order that the invention may be readily understood and put into practical effect, reference will now be made to embodiments of the present invention with reference to the accompanying drawings, wherein like reference numbers refer to identical elements. The drawings are provided by way of example only, wherein:

FIG. 1 shows the results obtained for ERG retinal function of hemp-injected animals compared to dim-reared and PBS-injected controls. (A-B) Hemp-injected animals had a significantly reduced a- and b-wave when compared to both dim-reared controls and PBS-injected controls after 5 days of PD (*P<0.05, two-way ANOVA with Sidak's post hoc test).

FIG. 2 shows the results obtained for the histological analysis of hemp-injected animals compared to PBS controls. (A-B) Photoreceptor cell death was increased in hemp-injected animals as evidenced by a lower number of photoreceptor rows and higher TUNEL+ cell counts. (C) There were no differences seen in IBA1 counts. (D-G) Representative images of TUNEL (red) and IBA1 (green) with DAPI (blue) as a nuclei stain (*P<0.05, Student's t test, scale bars indicate 50 μm).

FIG. 3 shows the results obtained for ERG retinal function of hemp-injected animals compared to PBS-injected controls. (A-B) No changes were observed in a- or b-waves in hemp-injected animals following 2 weeks of holding in dim cyclic light conditions when compared to PBS controls (P>0.05, two-way ANOVA with Sidak's post hoc test).

FIG. 4 shows the results obtained for ERG retinal function of hemp-injected animals compared to PBS-injected controls. (A) No changes in a-wave observed. (B) Hemp-injected animals had a significantly reduced b-wave when compared to PBS-injected controls. (C) 5 out of the 6 hemp-injected animals developed a cloudy cataract/eye infection in the eye as seen in these representative photos (*P<0.05, two-way ANOVA with Sidak's post hoc test).

Skilled addressees will appreciate that elements in the drawings are illustrated for simplicity and clarity and have not necessarily been drawn to scale. For example, the relative dimensions of some elements in the drawings may be distorted to help improve understanding of embodiments of the present invention.

### DETAILED DESCRIPTION OF THE INVENTION

The mouse model used in the below examples develops a dry-AMD-like lesion, that results from exposure to bright light, and is mediated by the ‘complement pathway’ of the immune system—as occurs in human disease. This could be thought of as ‘sterile inflammation’ since it occurs substantially in the absence of (bacterial or viral) infection and without genetic modification of the animal's natural immune responses. The lesion is anatomically and immunologically consistent with the human condition, Macular Degeneration. Both Dry and Wet MD involve inflammation and neurodegenerative elements, however GA has dominant chronic inflammatory component (Penfold, Philip L., and Jan M. Provis. Macular degeneration. Springer Science & Business Media, 2004).

Embodiments of the present invention relate to a composition and method of treatment for age related macular degeneration (AMD). More particularly, this invention relates to a composition and method of treatment for age related macular degeneration comprising hemp, hemp oil; or a pharmaceutically effective extract thereof.

AMD is a medical condition which may result in blurred or no vision in the center of the visual field. While in the early stages of this disease, the progression sees a gradual worsening of vision that may affect one or both eyes. Although it does not cause complete blindness, the resultant loss of central vision can make it difficult to recognize faces, drive, read, perform other daily activities and can reduce quality of life.

As used herein AMD includes Geographic Atrophy (GA). GA may also be known as atrophic age-related macular degeneration (AMD) or advanced AMD, which is an advanced form of age-related macular degeneration that can result in the progressive and irreversible loss of retina (photoreceptors, RPE and choriocappillaris).

Herein “AMD” is used to refer to dry AMD, GA and atrophic AMD.

Currently, more vision is lost to the dry form of AMD than the wet form. A long felt want exists for a treatment for AMD.

The inventor recognises that the dry lesion of AMD is regarded as the natural end stage of Macular Degeneration in the absence of neovascularisation. This means that in a significant number of cases the eye is dry rather than wet.

The data presented herein generated on a rodent (both mouse and rat models exist) model of AMD, which is published and recognised. The model is recognised and a number of therapeutic interventions have been accomplished in the model and show the model is pertinent and maps onto a human model and/or has parallels with the human condition).

While not wanting to be bound by any one theory, the inventor hypothesises that one or more of the pharmaceutically effective components of hemp are efficacious in the treatment of AMD. For this reason, the inventor has proposed that AMD may be effectively treated with hemp, hemp oil; or a pharmaceutically effective extract thereof.

The invention provides a method of treating or preventing AMD in a subject, the method comprising administering to the subject a therapeutically effective amount of hemp, hemp oil or pharmaceutically effective extract thereof to thereby treat or prevent the AMD. A pharmaceutical composition is also provided comprising a therapeutically effective amount of hemp, hemp oil; or a pharmaceutically effective extract thereof and a pharmaceutically acceptable carrier, diluent or excipient when used to treat AMD, as well as use of the pharmaceutical composition.

From the teaching herein a skilled person is readily able to select a suitable source for the low tetrahydrocannabinol (THC) hemp, hemp oil or pharmaceutically effective extract thereof. A suitable strain comprise Cannabis Ruderalis.

From the teaching herein a skilled person is readily able to select suitable dosages for the hemp, hemp oil; or pharmaceutically effective extract thereof.

The composition may comprise a water soluble dosage form.

The dosage form preferably comprises a form suitable for administration by one or more of intradermal, intramuscular, intraperitoneal, parenteral, intravenous, subcutaneous, intranasal, epidural, oral, sublingual, intracerebral, intravaginal, transdermal (e.g., via a patch), rectal, by inhalation, transmucosal, or topical, particularly to the ears, nose, eyes, or skin. The pharmaceutical composition may be injectable. The oral form may comprise a powder, a granule, a tablet, a capsule, a liquid, a suspension, an emulsion, a gel or a syrup. The parenteral or injectable form may comprise any suitable form for parenteral or injectable administration such as an injectable solution, an injectable suspension, an injectable emulsion, and an injection in a form that is prepared at the time of use. Formulations for parenteral administration may be in a configuration such as an aqueous or nonaqueous isotonic aseptic solution or suspension. The injectable form may be for intravitreal injection. The powder or liquid may be added to food or liquid for consumption.

The pharmaceutical composition of the invention may comprise a sustained release composition.

The method may further comprise administering to the subject at least one additional agent such as one or more Omega-3 Fatty Acid.

“Prevention” or “prophylaxis,” as used herein, refers to prophylactic or preventative measures. Those in need of prevention or prophylaxis include those in whom the AMD is to be prevented, and in some embodiments, may be predisposed or susceptible to the eye disease or condition e.g. individuals with a family history of an eye disease or condition.

Prevention or prophylaxis is successful herein if the development of AMD is completely or partially prevented or slowed down.

“Treatment” of a subject herein refers to therapeutic treatment. Those in need of treatment include those already with AMD, as well as those in whom the progress of AMD is to be prevented. Hence, the subject may have been diagnosed as having AMD or may have AMD or damage that is likely to progress in the absence of treatment. Alternatively, the subject may be symptom-free, but has risk factors for development of AMD e.g., positive family history. Treatment is successful herein if the AMD is alleviated or healed, or progression of the AMD, including its signs and symptoms and/or structural damage, is halted or slowed down as compared to the condition of the subject prior to administration. Successful treatment further includes complete or partial prevention of the development of the AMD. For purposes herein, slowing down or reducing the AMD or the progression of the AMD is the same as arrest, decrease, or reversal of the AMD.

The expression “effective amount” refers to an amount of an agent or medicament, either in a single dose or as part of a series, which is effective for treating or preventing AMD or predisposition thereto. This would include an amount that is effective in achieving a reduction in one or more symptom as compared to baseline prior to administration of such amount as determined, e.g., by visual acuity or other testing. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

The terms “subject”, “patient” or “individual,” which are used interchangeably herein, refer to any subject, particularly a vertebrate subject, and even more particularly a mammalian subject, for whom therapy or prophylaxis is desired. Suitable vertebrate animals that fall within the scope of the invention include, but are not restricted to, any member of the subphylum Chordata including humans, as well as non-human primates, rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (snakes, frogs, lizards etc.), and fish. In specific embodiments, the “subject”, “patient” or “individual” is a human in need of treatment or prophylaxis of an eye disease or condition, including in subjects with a diabetic eye disease or condition or an ocular tumour. In specific embodiments, the terms “subject”, “patient” or “individual” refer to any single human subject, including a patient, eligible for treatment who is experiencing or has experienced one or more signs, symptoms, or other indicators of AMD or predisposition thereto, whether, for example, newly diagnosed or previously diagnosed and now experiencing a recurrence or relapse, or is at risk for AMD, no matter the cause. Intended to be included as a “subject”, “patient” or “individual” are any subjects involved in clinical research trials not showing any clinical sign of disease, or subjects involved in epidemiological studies, or subjects once used as controls. The “subject”, “patient” or “individual” may have been previously treated with a medicament for AMD, or not so treated.

As used herein, a derivative includes a therapeutically active or pharmaceutically active fragment of a compound modulating the activity of a mineralocorticoid receptor or a glucocorticoid receptor.

An analog may be a structural analog or a functional analog.

A homolog may comprise a molecule of the same chemical type, but differing by a fixed increment of an atom or a constant group of atoms. An example is methyl and ethyl alcohols which are homologous.

Table 1 below shows some example compounds and their measured mineralcorticoid and glucocorticoid properties.

In one embodiment, the compositions of the invention comprise a sustained release composition. Based on the teachings herein, a skilled person is readily able to select and/or formulate a suitable sustained release composition.

In another embodiment, the compositions and components thereof may be sterilised. From the teachings herein, a skilled person is readily able to select a suitable sterilisation method such as, heat treatment.

In another embodiment, the compositions of the invention are preservative free.

By “pharmaceutically-acceptable carrier, diluent or excipient” is meant a solid or liquid filler, diluent or encapsulating substance that may be safely used in systemic administration. Depending upon the particular route of administration, a variety of carriers, well known in the art may be used. These carriers may be selected from a group including sugars, starches, cellulose and its derivatives, malt, gelatine, talc, calcium sulfate, vegetable oils, synthetic oils, polyols, alginic acid, phosphate buffered solutions, emulsifiers, isotonic saline and salts, such as mineral acid salts including hydrochlorides, bromides and sulfates, organic acids such as acetates, propionates and malonates and pyrogen-free water.

The one or more pharmaceutically acceptable carriers, diluents or excipients may comprise one or more of a wetting agent and a viscosity modifier.

A useful reference describing pharmaceutically acceptable carriers, diluents and excipients is Remington's Pharmaceutical Sciences (Mack Publishing Co. N.J. USA, 1991), which is incorporated herein by reference.

The above compositions may be administered in a manner compatible with the dosage formulation, and in such amount as is pharmaceutically-effective. The dose administered to a patient, in the context of the present invention, should be sufficient to effect a beneficial response in a patient over an appropriate period of time. The quantity of agent(s) to be administered may depend on the subject to be treated inclusive of the age, sex, weight and general health condition thereof, factors that will depend on the judgement of the practitioner.

The following non-limiting examples illustrate the invention. These examples should not be construed as limiting: the examples are included for the purposes of illustration only. The Examples will be understood to represent an exemplification of the invention.

### EXAMPLES

### Example 1

### METHODS

### Cannabis Studies:

A mouse model of dry AMD was used in a controlled study, with and without hemp oil to see any therapeutic effect. The hemp oil is to be presented in an aqueous suspension. The whole oil will be used initially. Subsequent investigation may reveal efficacious components.

Hemp extracts may be neuroprotective including the retina. The retina may benefit from neuroprotective compounds such as, those known to treat epilepsy. Fish oils, in particular omega oils, are known to be good for blood supply. While not wanting to be bound by any one theory the inventor's rationale is to develop pro-vasculature, pro-angiogenic agents.

### Animals and Light Exposure:

All experiments conducted were in accordance with the ARVO Statement for the Use of Animals in Ophthalmic and Vision Research. Albino Sprague-Dawley (SD) rats aged from 130 to 160 postnatal days were exposed to bright continuous light (BCL) at 1000 lux. The rats were born and reared in dim cyclic light conditions (12 h light:12 h dark) with an ambient light level of approximately 5 lux. Exposure to BCL was conducted on animals aged between post-natal days (P) 90-150. Prior to BCL exposure, rats were dark adapted for a minimum of 15 h then transferred to individual cages designed to allow light to enter unimpeded. There were no areas of shadow in the cages; pupillary dilation was not performed. BCL exposure commenced consistently at 9:00 am, and was achieved using a cold-white fluorescent light source positioned above the cages (18W, Cool White; TFC), at an intensity of approximately 1000 lux at the cage floor. BCL exposure was maintained over a period of 24 h, after which time the animals were immediately returned to dim cyclic conditions for the post-exposure period. Animals were kept in dim light conditions following BCL exposure for a maximum period of 56 days.

The animals were exposed to BCL for a period of 1, 3, 6, 12, 17, or 24 hours, after which time retinal tissue was obtained for analysis. Some animals were returned to dimlight (5 lux) conditions immediately after 24 hours of BCL for a period of 3 or 7 days, to assess postexposure effects. Age-matched, dim-reared animals served as control samples.

### Tissue Collection and Processing

Animals were euthanatized by overdose of barbiturate administered by an intraperitoneal injection (60 mg/kg bodyweight; Valabarb; Virbac Animal Health, Regents Park, NSW, Australia). The left eye from each animal was marked at the superior surface for orientation and then enucleated and processed for cryosectioning, and the retina from the right eye was excised through a corneal incision and prepared for RNA extraction.

### Microarray Experimentation and Analysis

Microarray analysis was performed using raw microarray data derived from a previous study (Natoli R, Zhu Y, Valter K, Bisti S, Eells J, Stone J. Gene and noncoding RNA regulation underlying photoreceptor protection: microarray study of dietary antioxidant saffron and photobiomodulation in rat retina. Mol Vis. 2010; 16:1801-1822) using rat gene microarrays (Rat Gene 1.0 ST; Affymetrix, Santa Clara, Calif.). The full set of microarray data has been deposited in the NCBI Gene Expression Omnibus repository under accession number GSE22818 (National Center for Biotechnology Information, National Institutes of Health, Bethesda, Md.). The analysis compared samples from dimreared and 24-hour BCL experimental groups (n=3 for each). The microarray data were analyzed (Partek Genomics Suite 6.4 software; Partek Inc., St. Louis, Mo.), and CEL files (Affymetrix) were imported into the software with background correction, normalization, and summarization, using the robust multiarray average (RMA) algorithm adjusted for probe sequence and GC content (GC-RMA). The processed values were displayed as individual probe sets representing exonic coding sequences, which were log-transformed using base 2. Differential expression analysis was performed using the analysis of variance (ANOVA) statistic with significance level of P″0.05. The heterogeneity of the resulting differential expression data was evaluated with agglomerative hierarchical clustering, using the Euclidean distance metric and principle component analysis (PCA; both provided by the Genomics Suite; Partek). The differential expression data were then clustered according to biological process as described by the Gene Ontology Consortium, using functional analysis with Gene Ontology (GO) enrichment provided by the software (Partek GS Genomics Suite). After this, the list of differentially expressed genes was screened for those relating to the complement cascade, using a differential expression cutoff of #50% and aided by pathway information summarized from the Gene Ontology Consortium and gene grouping from the HUGO Gene Nomenclature Committee.

Quantitative Real-Time Polym erase Chain Reaction

First-strand cDNA synthesis was performed as (SuperScript III Reverse Transcriptase kit, cat. no. 18080-044; Invitrogen) according to the manufacturer's instructions. A 20-μL reaction mixture was used in conjunction with 1 μg RNA, 500 ng oligo (dT)18 primer, and 200 U reverse transcriptase. Gene amplification was measured using either commercially available hydrolysis probes (TaqMan; Applied Biosystems, Inc. [AM], Foster City, Calif.) or SYBR Green with custom designed primers, the details of which are provided in Tables 2 and 3, respectively. The hydrolysis probes were applied according to a previously established qPCR protocol. The primers for SYBR Green qPCR (Table 3) were designed within a coding domain sequence transversing an intron using the Primer3 web-based design program. The qPCR was performed using a commercial qPCR system (StepOnePlus; ABI). The amplification for each biological sample was performed in experimental triplicate, with the mean Cq (quantitation cycle) value then used to determine the ratio of change in expression. For both qPCRs (Taqman and SYBR Green; ABI), the percentage change compared to dim-reared samples was determined using the Cq method. The expression of the target gene was normalized to the expression of the reference gene glyceralde-hyde-3-phosphate dehydrogenase (GAPDH), which showed no differential expression in the present study or in previous light-induced retinal damage investigations. Amplification specificity was assessed using gel electrophoresis. Statistical analysis was performed using the one-way ANOVA, to assess the significance of the trend in expression. Differences with a P″0.05 were considered statistically significant.

### Example 2

### Methods

### Animals

### High CBD Low TCH Hemp Oil

All experiments using hemp were performed under the ACT Health Prohibited Substance Research and Education Program Licence S9-0035/18. Hemp extract in hemp seed oil was at a concentration of 30 mg/ml (CBD 3%, THC 0%, Drug Control Section import permit CSH1815969). The hemp extract was manufactured on Dec. 18, 2012 with an expiry date of December 2020. Hemp was stored at room temperature (<25° C.), protected from light, excessive heat and moisture and stored in a high-density polyethylene (HDPE) jug.

### Intravitreal Injections

Prior to all intravitreal injections, animals were restrained and anaesthetized using a mixture of Ketamine (100 mg/kg body weight; Troy Laboratories, NSW, Australia) and Ilium Xylazil-20 (12 mg/kg body weight; Troy Laboratories), delivered through an intraperitoneal injection. A pupil dilator was administered to both eyes (Minims Atropine Sulphate 1% w/v eye drops; Bausch and Lomb, NSW, Australia). Following dilation of the pupil, a cotton string loop was tied around the eye, and 10% w/v povidone-iodine antiseptic liquid (Betadine; Faulding Pharmaceuticals, SA, Australia) was applied to each eye before injection. Using a 30 G beveled needle (Becton Dickinson, N.J., USA), a small puncture wound was made into the eye approximately 1 mm from the limbus. Hemp extract oil was injected using a 10 μl Hamilton syringe with an attached 34 G beveled needle (World Precision Instruments, FL, USA). Endotoxin-free 0.1 M PBS (pH 7.4, Thermo Fisher Scientific, Waltham, Mass.) was injected for control animals. 1 μl of respective solution was injected into each eye. Chlorsig antibiotic cream (Aspen Pharma, NSW, Australia) was applied to the injection site. Eye gel (GenTeal; Novartis, NSW, Australia) was administered to both eyes to prevent dryness, and the animal was recovered on a heat mat to maintain core body temperature.

### Photo-Oxidative Damage (PD)

Mice were placed into Perspex boxes coated with a reflective interior surface and exposed to 100 K lux of natural white light-emitting diodes (LED) for 5 days, with free access to food and water. Lighting levels were set to 100 K lux using a light meter device (HD450; Extech MA, USA). During the course photo-oxidative damage, each animal was administered with pupil dilator eye drops twice daily, morning and evening (Minims Atropine Sulphate 1% w/v eye drops).

### Electroretinography (ERG)

Following PD, electroretinography was used to measure mouse retinal function in response to full-field flash stimuli under scotopic conditions. Mice were dark-adapted overnight prior to the measurement of retinal recordings. Prior to ERG recordings animals were restrained and anaesthetized using a mixture of Ketamine (100 mg/kg body weight; Troy Laboratories, NSW, Australia) and Ilium Xylazil-20 (12 mg/kg body weight; Troy Laboratories), delivered through an intraperitoneal injection. Mice were administered with a pupil dilator to both eyes, phenylephrine hydrochloride (w/v 2.5%, Bausch and Lomb) followed by tropicamide (1.0% w/vBausch and Lomb). Mice were placed on the Celeris ERG heatpad (Celeris, Diagnosys, MA, USA). A drop of hypermellose solution (0.3% w/v Genteal, Bausch and Lomb) was placed onto each eye and the corneal probes positioned to encapsulate the eyes (both eyes recorded at the same time). Animals were subjected to a series of flashes up to 10 cds.m² with a-wave (photoreceptor) and b-wave (second order neurons) responses measured from both eyes. Following data collection, animals were recovered on a heat mat to maintain core body temperature.

### Tissue Collection and Processing

Animals were culled with carbon dioxide (CO₂) exposure. Whole eyes were excised for histological analyses whereas retinas were taken for RNA extraction. To extract the whole eye, the eye was lifted from the socket by placing curved forceps (World Precision Instruments) underneath the eye lid closing in on the optic nerve and connecting tissues. The superior surface of the eye was marked with a permanent marker pen for orientation. A pair of ophthalmic scissors (World Precision Instruments) was inserted beneath the forceps cutting the eye from the socket. The eye was immediately injected with approximately 20 μl of 4% paraformaldehyde using a 34G insulin syringe beneath the limbus in the sclera. The excised eye was then immersed in 4% paraformaldehyde for 4 hours.

Enucleated eyes are fixed in 4% paraformaldehyde for 4 hours. Following this, they are washed three times in 0.1 M PBS and immersed into a 15% sucrose solution overnight at 4° C. Truncated plastic embedding moulds (Pro Sci Tech, QLD, Australia) were filled with the Tissue-Tek compound (Sakura Finetek USA Inc., CA, USA) and each eye was placed into an individual mould, taking care in orientating the superior surface of the eye in the same direction for each eye. The eyes are then embedded in Tissue-Tek Optimal Cutting Temperature Compound, and snap frozen in an acetone/dry ice mixture that is at approximately 78° C. The moulds are then wrapped in aluminium foil and transferred to −20° C. until needed for cryosectioning.

### Cryosectioning

A Leica CM 1850 cryostat (Leica Microsystems) was used to cut retinal cryosections from the snap-frozen eyes at −22° C. Cryosections are cut at 16 μm for rat eyes, and 12 μm for mouse eyes. Each eye was mounted onto the cryostat chuck orientated using the superior side to ensure the same direction for all sections. Eyes were sectioned in the para-sagittal plane. Cryosections from the optic nerve central region of the eye were collected on Superfrost Ultra Plus glass slides (Menzel-Glaser, Braunschweig, Germany) in duplicate, in order for analysis of the localised region of damage in the superior area centralis of the animal retin. Slides were oven-dried at 37° C. overnight before being transferred to −20° C. indefinitely until needed for histological staining and analyses.

### Histological Analyses

The terminal deoxynucleotidyl transferase (Tdt) dUTP nick end-labelling (TUNEL) assay was used on retinal cryosections to detect photoreceptor cell death. This was performed using a Tdt enzyme (Roche Diagnostics, Basel, Switzerland), biotin-dUTP (Roche Diagnostics) and a Streptavidin-Alexa Fluor-594 secondary label (Thermo Fisher Scientific, MA, USA). Sections were counterstained using a DNA label (Bisbenzimide or Hoechst's stain, 1:10,000; Sigma Aldrich) for visualisation of the cellular layers.

The detection and localisation of microglia/macrophages in situ was determined via immunohistochemistry (IHC) against IBA1 (1:500 dilution, Wako, Osaka, Japan). Antigen retrieval (ImmunoSolutions, QLD, Australia) was performed on retinal cryosections prior to incubation with primary antibodies overnight (16 hours) at 4° C. Sections were then incubated with secondary antibodies, conjugated to either Alexa 488 (1:1000; Thermo Fisher Scientific) for 4 hours at room temperature. Slides were counterstained with a DNA label.

TUNEL and IBA1 positive cells were quantified along the full length of retinal cryosections (supero-inferior) in duplicate. Outer retinal counts (ONL-RPE) were taken for IBAI+ staining. To determine photoreceptor cell death, only TUNEL+ cells in the ONL were quantified. To quantify photoreceptor loss in retinal cryosections, the number of rows of photoreceptor nuclei (visualised using a DNA label) was quantified at the lesion site in the superior retina (approximately 1 mm superior to the optic nerve). For each section, 3 measurements were taken, and each eye was counted in duplicate.

### Imaging and Statistics

Fluorescence in retinal cryosections was visualised and imaged using a laser-scanning A1+ confocal microscope (Nikon, Tokyo, Japan). Images were acquired using the NIS-Elements AR software (Nikon). Negative control slides were visualised and imaged with each different immuno-label, to determine specificity of staining and the lower threshold of fluorescence detection for comparison to positively-stained slides. All slides were imaged using the same settings for each immuno-label for the comparison of positive staining between experimental groups. Images were assembled into figure panels using Photoshop CS6 software (Adobe Systems, CA, USA). All graphing and statistical analysis was performed using Prism 6 (GraphPad Software, CA, USA), using unpaired Student's t-tests, one-way analysis of variance (ANOVA) or two-way ANOVA with the appropriate post-hoc tests to determine statistical significance (P<0.05). Graphs were generated with mean+SEM values.

### Results

Functional Analysis Post-Hemp Injection in the Retina after 5 Days of PD

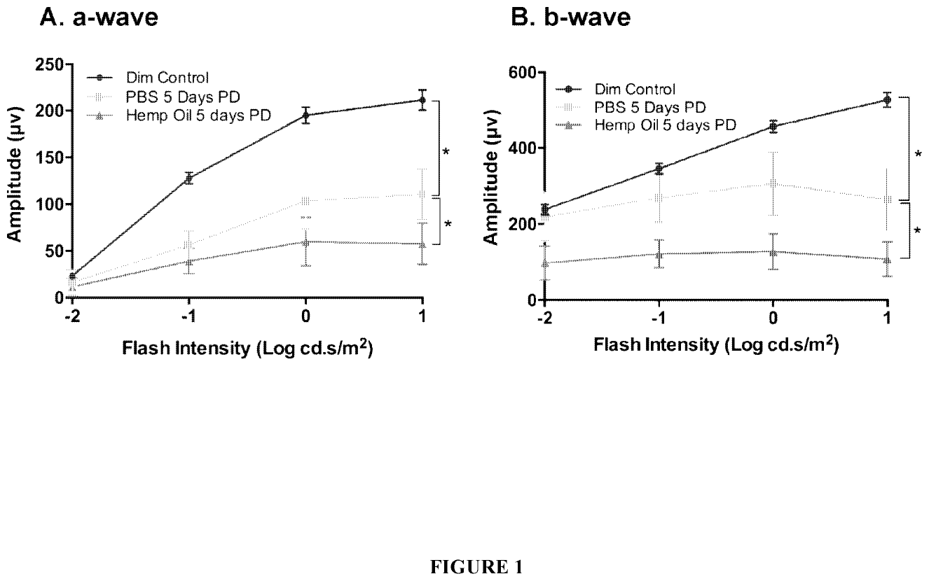

The effect of hemp on retinal degeneration was assessed using the PD model. Following intravitreal injections of PBS or hemp, and 5 days of PD, retinal function was measured using ERG analysis. Following PD, the animals injected with hemp were found to have a decreased ERG retinal function in both the a- and b-waves when compared with PBS controls (P<0.05, FIGS. 1A and B).

Histological Analysis Post-Hemp Injection in the Retina after 5 Days of PD

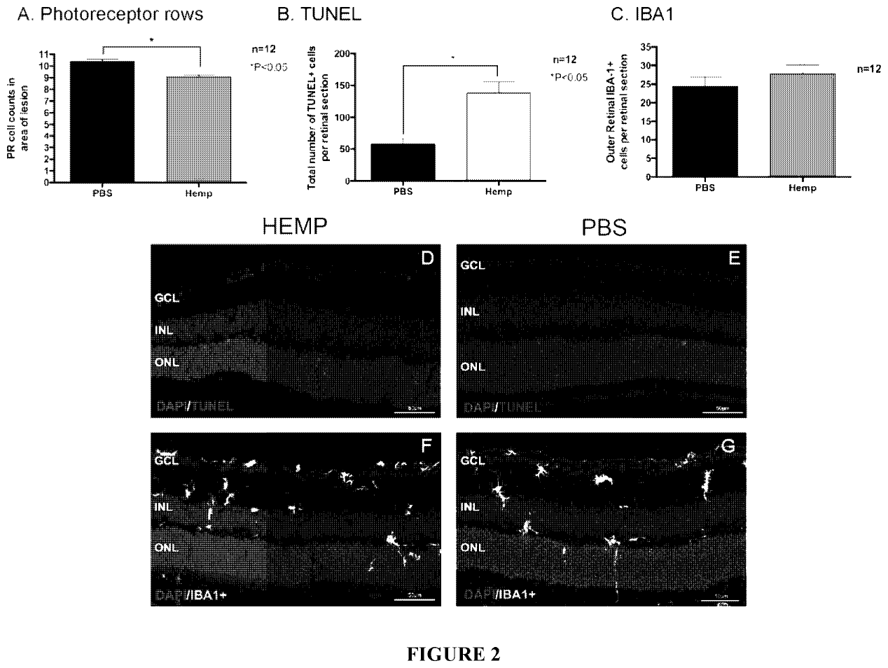

Retinal tissue was collected from these animals for histological analysis of retinal damage. TUNEL staining for photoreceptor cell death and photoreceptor row measurements were used to assess photoreceptor cell loss at 5 days of PD. The number of photoreceptor rows in the ONL at the area of highest damage in central retina (1 mm superior to the optic nerve) was counted to assess cumulative photoreceptor layer thinning over the 5 days. It was found that there was a significant decrease in the amount of photoreceptor rows in hemp-injected animals indicating a higher amount of photoreceptor loss when compared with PBS controls (P<0.05, FIG. 2A). There was a significant increase in the amount of TUNEL+ photoreceptors undergoing cell death in animals injected with hemp oil compared to the PBS controls (P<0.05, FIG. 2B, D, E).

The contribution of microglia and macrophages in the outer retina to retinal damage was investigated following 5 days of PD. This was assessed using IBA1 immunohistochemistry. It was found that there was no significant different in IBA1+ microglia/macrophage numbers in the outer retina between the PBS and hemp animals (P>0.05, FIG. 2C, F, G).

### Functional Analysis 2 Weeks Post-Hemp Injection (Pre- and Post-5 Days PD)

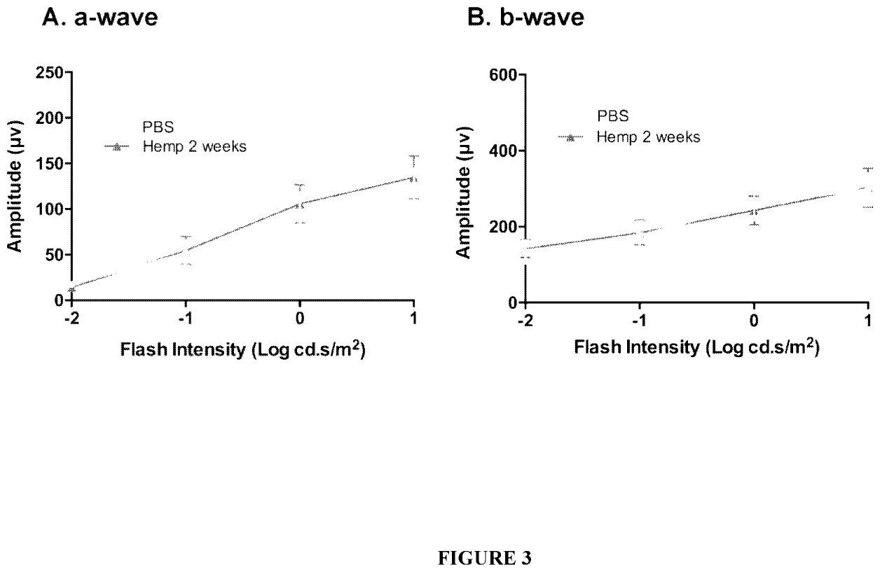

At 2 weeks post-injection, there was no significant difference in ERG function between hemp and PBS controls in both a-wave and b-waves (P>0.05, FIG. 3A-B).

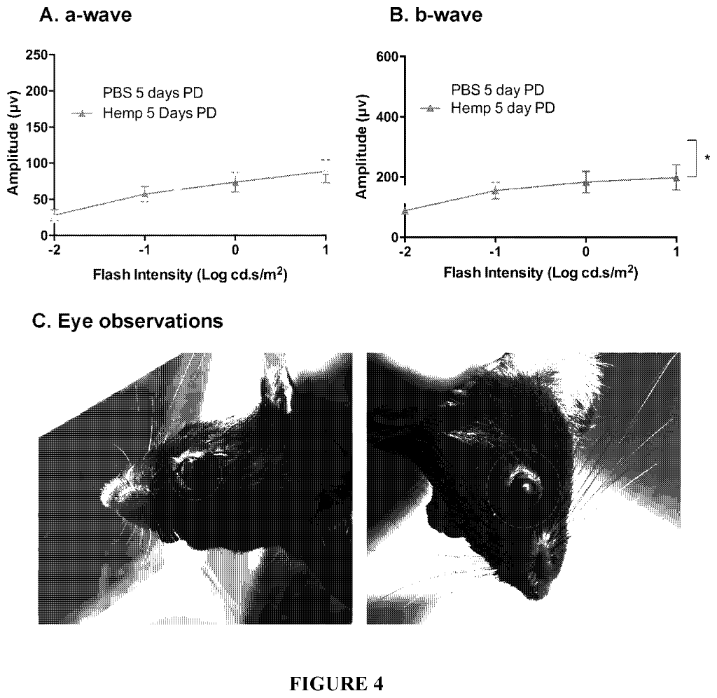

After 5 days of PD, 2 weeks post-injection, no changes in the a-wave were observed (P>0.05, FIG. 4A). However, there was a significant decrease in ERG function for the b-wave of hemp animals compared with PBS controls (P<0.05, FIG. 4B). Additionally, 5 of the 6 animals injected with hemp oil developed cataracts/eye infections throughout the PD time course (FIG. 4C).

### CONCLUSIONS

This data indicates that intravitreal delivery of hemp oil did not result in any substantially adverse functional effects on the retina at 2 weeks post-injection as measured via ERG. However, in both photo-oxidative damage paradigms (0 day and 2 weeks post-injection), significant retinal damage was observed in those animals injected with hemp oil.

Hemp oil was intravitreally delivered prior to 5 days of PD. This data shows that these animals had a significantly decreased retinal function and increase in photoreceptor cell death when compared to controls only injected with PBS. This indicates an appreciable amount of retinal damage after hemp oil is delivered to the retina, followed by subsequential retinal stress in the form of photo-oxidative damage.

In light of the above, intravitreal injections of hemp oil were considered to result in significant retinal damage following the onset of a stress stimulus to the eye. However, stand-alone hemp injections without subsequent induced retinal degeneration did not appear to affect the retinal function of the animals.

In this specification, the terms “comprises”, “comprising” or similar terms are intended to mean a non-exclusive inclusion, such that an apparatus that comprises a list of elements does not include those elements solely, but may well include other elements not listed.

Throughout the specification the aim has been to describe the invention without limiting the invention to any one embodiment or specific collection of features. Persons skilled in the relevant art may realize variations from the specific embodiments that will nonetheless fall within the scope of the invention.

### Tables

TABLE 1
Mineralocorticoid Receptor and Glucocorticoid Receptor activity of some
corticosterones

| | | | Duration of |
|---|---|---|---|
| | | | action |
| Compound | GR potency | MR potency | (t_{1/2} in hours) |
| Hydrocortisone | 1 | 1 | 8 |
| (cortisol) | | | |
| Cortisone | 0.8 | 0.8 | oral 8; |
| | | | i.m. 18+ |
| Prednisone | 3.5-5 | 0.8 | 16-36 |
| Prednisolone | 4 | 0.8 | 16-36 |
| Methylprednisolone | 5-7.5 | 0.5 | 18-40 |
| Dexamethasone | 25-80 | 0 | 36-54 |
| Betamethasone | 25-30 | 0 | 36-54 |
| Triamcinolone | 5 | 0 | 12-36 |
| Beclometasone | 8 puffs 4 times | — | — |
| | a day; equals 14 mg | | |
| | oral prednisone | | |
| | once/day | | |
| Fludrocortisone | 15 | 200 | 24 |
| acetate | | | |
| Deoxycorticosterone | 0 | 20 | — |
| acetate (DOCA) | | | |
| Aldosterone | 0.3 | 200-1000 | — |

Key:
MR = mineralocorticoid receptor;
GR = glucocorticoid receptor;
i.m. intramuscular

TABLE 2
Genetic Analysis Probes

| [symbol] | [symbol] | [symbol] | [symbol] |
|---|---|---|---|
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |
| [symbol] | [symbol] | [symbol] | [symbol] |

[symbol]

[symbol]

indicates data missing or illegible when filed

TABLE 3
qRT PCT Custom Primer Sets

| [symbol] | [symbol] | [symbol] | [symbol] |
|---|---|---|---|
| [symbol] | [symbol] | [symbol] | [symbol] |
| [symbol] | [symbol] | [symbol] | [symbol] |

[symbol]

indicates data missing or illegible when filed

1. A method of treating or preventing AMD in a subject, the method comprising administering to the subject a therapeutically effective amount of hemp, hemp oil or pharmaceutically effective extract thereof to thereby treat or prevent the AMD.

2. A pharmaceutical or composition comprising a therapeutically effective amount of hemp, hemp oil; or a pharmaceutically effective extract thereof and a pharmaceutically acceptable carrier, diluent or excipient when used to treat AMD.

3. (canceled)

4. The method of claim 1, wherein the hemp, hemp oil or pharmaceutically effective extract thereof comprises a compound selected from a cannabinoid, a Non-Steroidal Anti-Inflammatory Drug (NSAID), ibuprofen, copper ibuprofenate, indomethacin, copper indomethacin, naproxen, flurbiprofen, celecoxib, aceclofenac, acemetacin, acetylsalicylic acid, 5-amino-acetylsalicylic acid, alclofenac, alminoprofen, amfenac, bendazac, bermoprofen, alpha-bisabolol, bromfenac, bromosaligenin, bucloxic acid, butibufen, carprofen, cinmetacin, clidanac, clopirac, diclofenac sodium, diflunisal, ditazol, enfenamic acid, etodolac, etofenamate, felbinac, fenbufen, fenclozic acid, fendosal, fenoprofen, fentiazac, fepradinol, flufenamic acid, flunixin, flunoxaprofen, flurbiprofen, glucametacin, glycol salicylate, ibuprofen, ibuproxam, indomethacin, indoprofen, isofezolac, isoxepac, isoxicam, ketoprofen, ketorolac, lornoxicam, loxoprofen, meclofenamic acid, mefenamic acid, meloxicam, mesalamine, metiazinic acid, mofezolac, naproxen, niflumic acid, oxaceprol, oxaprozin, oxyphenbutazone, parsalmide, perisoxal, phenyl acetylsalicylate, olsalazine, pyrazolac, piroxicam, pirprofen, pranoprofen, protizinic acid, salacetamide, salicilamide O-acetic acid, salicylsulphuric acid, salsalate, sulindac, suprofen, suxibuzone, tenoxicam, tiaprofenic acid, tiaramide, tinoridine, tolfenamic acid, tolmetin, tropesin, xenbucin, ximoprofen, zaltoprofen, zomepirac, tomoxiprol; sulindac, 11-desoxycortisone (11-DC), fludrocortisone, fludrocortisone acetate (FA), fludrocortisone acetonide; Deoxycorticosterone acetate (DA), Deoxycorticosterone (DS), Aldosterone, cortisol, cortisone, prednisone, prednisolone, methylprednisolone, dexamethasone, betamethasone, triamcinolone, triamcinolone acetonide, beclomethasone, or a therapeutically active analogue, derivative, homolog, pharmaceutically acceptable salt or conjugate thereof.

5. The method of claim 4, wherein the cannabinoid comprises cannabidiol.

6. The method of claim 1, wherein the hemp, hemp oil or pharmaceutically active extract thereof comprises a low Tetrahydrocannabinol (THC) hemp, hemp oil or pharmaceutically effective extract thereof.

7-8. (canceled)

9. The method of claim 1, wherein the hemp, hemp oil, or therapeutically effective amount or pharmaceutical composition comprises a form suitable for administration by one or more of oral intradermal, intramuscular, intraperitoneal, parenteral, intravenous, subcutaneous, intranasal, epidural, sublingual, intracerebral, intravaginal, transdermal, rectal, by inhalation, transmucosal, or topical.

10-16. (canceled)

17. The method of claim 1, further comprising administering to the subject at least one additional agent.

18. The method of claim 1, wherein the additional agent comprises one or more Omega Fatty Acids.

19. The method of claim 1, wherein the pharmaceutical composition is a delivery vehicle for one or more compounds.

20. The method of claim 19, wherein the one or more compounds is selected from a cannabinoid, a Non-Steroidal Anti-Inflammatory Drug (NSAID), ibuprofen, copper ibuprofenate, indomethacin, copper indomethacin, naproxen, flurbiprofen, celecoxib, aceclofenac, acemetacin, acetylsalicylic acid, 5-amino-acetylsalicylic acid, alclofenac, alminoprofen, amfenac, bendazac, bermoprofen, alpha-bisabolol, bromfenac, bromosaligenin, bucloxic acid, butibufen, carprofen, cinmetacin, clidanac, clopirac, diclofenac sodium, diflunisal, ditazol, enfenamic acid, etodolac, etofenamate, felbinac, fenbufen, fenclozic acid, fendosal, fenoprofen, fentiazac, fepradinol, flufenamic acid, flunixin, flunoxaprofen, flurbiprofen, glucametacin, glycol salicylate, ibuprofen, ibuproxam, indomethacin, indoprofen, isofezolac, isoxepac, isoxicam, ketoprofen, ketorolac, lornoxicam, loxoprofen, meclofenamic acid, mefenamic acid, meloxicam, mesalamine, metiazinic acid, mofezolac, naproxen, niflumic acid, oxaceprol, oxaprozin, oxyphenbutazone, parsalmide, perisoxal, phenyl acetylsalicylate, olsalazine, pyrazolac, piroxicam, pirprofen, pranoprofen, protizinic acid, salacetamide, salicilamide O-acetic acid, salicylsulphuric acid, salsalate, sulindac, suprofen, suxibuzone, tenoxicam, tiaprofenic acid, tiaramide, tinoridine, tolfenamic acid, tolmetin, tropesin, xenbucin, ximoprofen, zaltoprofen, zomepirac, tomoxiprol; sulindac, 11-desoxycortisone (11-DC), fludrocortisone, fludrocortisone acetate (FA), fludrocortisone acetonide; Deoxycorticosterone acetate (DA), Deoxycorticosterone (DS), Aldosterone, cortisol, cortisone, prednisone, prednisolone, methylprednisolone, dexamethasone, betamethasone, triamcinolone, triamcinolone acetonide, beclomethasone, or a therapeutically active analogue, derivative, homolog, pharmaceutically acceptable salt or conjugate thereof.

21. The method of claim 20, wherein the one or more compounds comprises an hydrophobic compound.

22. The method of claim 1, wherein the method comprises administering to the subject a therapeutically effective amount of hemp oil and the hemp oil is hemp seed oil.

23. The method of claim 22, wherein the hemp seed oil is cold pressed hemp seed oil.

24. The method of claim 1, wherein the hemp oil has a ratio of Omega 3 to Omega 6 of between about 1:5.2 and 5:16 or a ratio of Omega 3 to Omega 6 of about 3.5:11.6.

25. The pharmaceutical composition or composition of claim 2, wherein the hemp, hemp oil or pharmaceutically active extract thereof comprises a low Tetrahydrocannabinol (THC) hemp, hemp oil or pharmaceutically effective extract thereof.

26. The pharmaceutical composition or composition of claim 2, wherein hemp oil is hemp seed oil.

27. The pharmaceutical composition or composition of claim 26, wherein the hemp seed oil is cold pressed hemp seed oil.

28. The pharmaceutical composition or composition of claim 2, wherein the hemp oil has a ratio of Omega 3 to Omega 6 of between about 1:5.2 and 5:16 or a ratio of Omega 3 to Omega 6 of about 3.5:11.6.
